(19) United States
(12) Patent Application Publication
(10) Pub. No.: US 2006/0013801 A1
(43) Pub. Date: Jan. 19, 2006

(54) Prevention of and therapy for radiation toxicity of normal tissues using drugs which block il-1 activity

(76) Inventors: Paul Okunieff, Rochester, NY (US); Ivan Ding, Rochester, NY (US); Yuhchyau Chen, Pittsford, NY (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/505,693

(22) PCT Filed: Feb. 25, 2003
(86) PCT No.: PCT/US03/05624

§ 371 Date: Jul. 15, 2005

Related U.S. Application Data
(60) Provisional application No. 60/360,093, filed on Feb. 25, 2002.

(51) Int. Cl. A61K 38/20; A61K 38/18

(52) U.S. Cl. 424/85.2; 514/12

(57) ABSTRACT

A method for the prevention of and therapy for radiation pneumonitis, dermatitis, soft tissue fibrosis and central nervous system toxicity in patients undergoing therapeutic radiation. In addition, the invention provides for pre-treatment of those responding to nuclear bio terrorism or other nuclear or radiological accidents. Thus, with the present invention, subjects may be treated in order to prevent toxicity from nuclear bio terrorism or other nuclear or radiological accidents. More particularly, we have discovered a method for prophylactically treating radiation toxicity in normal tissue of subject comprising administering an anti-radiation toxicity effective amount of a cytokine blocking agent through the subject. More specifically, we have discovered a method for prophylactically treating radiation pneumonitis, dermatitis, soft tissue fibrosis or central nervous system toxicity in a subject comprising administering an anti-radiation pneumonitis, dermatitis, soft tissue fibrosis or central nervous system toxicity effective amount of a cytokine blocking agent to the subject.

### BACKGROUND

There are few if any treatments for radiation exposure that have quantitative dose modifying benefits when given hours a day after exposure. Similarly, even those drugs that have benefits when given before radiation, typically are ethicacious only for a few hours and have transient side effects that prevent the subject from full function, for example, hypotension and peripheral neuropathy.

Radiation-induced soft tissue fibrosis is a consequence of acute and chronic inflammatory responses. While modern radiation techniques have improved therapeutic gain and reduced the incidence of severe radiation-induced fibrosis, radiation-related side effects still occur when aiming for optimal tumor control. It has been shown that radiation-induced soft tissue damage is expected in about 10% of patients when radiation is optimized to achieve 90% tumor control.

Soft tissue fibrosis occurs in the late stage of radiation-induced tissue damage. It is caused by multiple factors and is poorly understood. However, the early stage of radiation-induced soft tissue damage is characterized by infiltration of various inflammatory cells and overproduction of cytokines. The late stage is pathologically characterized by active fibroblast proliferation with atypical fibroblasts, and excessive extracellular matrix production. Radiation injury is similar in some ways to normal tissue injury. Surgical injury, for example, is a process that features a relatively short period of brisk cytokine production, angiogenesis, fibroblast, and epithelial cell proliferation. The atypical proliferation results in granulation, which abruptly stops, allowing mature scar to develop. IL-1 is an important signal controlling this process. Radiation-induced soft tissue fibrosis has many of the same features of normal tissue repair, but is less brisk and may remain active for years at subclinical levels. The continuous inflammation results in continuously active deposition of collagen.

Radiation pneumonitis is a distinct clinical entity that differs from other pulmonary symptoms such as allergic pneumonitis, chemical pneumonitis, or pneumonia by various infectious agents. Recent research has supported the mechanism of cellular interaction between lung parenchymal cells and circulating immune cells mediated through a variety of cytokines including pro-inflammatory cytokines, chemokines, adhesion molecules, and pro-fibrotic cytokines. Identifying reliable biomarkers for radiation pneumonitis will allow identifying individuals at risk for pneumonitis before or during the early stage of therapy.

Radiation pulmonary injury manifested as subacute pneumonitis and late fibrosis has long been recognized in patients receiving radiotherapy to the chest region. Lung injury by radiation is a major obstacle prohibiting the high dose radiation required for eradicating cancer of the thoracic region. Radiation pneumonitis is a distinct clinical entity and there has been increasing awareness and recognition of its impact on the treatment of thoracic malignancy. It manifests unique clinical and radiographic characteristics that separate it from other pulmonary symptoms such as allergic pneumonitis, chemical pneumonitis, or pneumonia by various infectious agents of viral, bacterial, fungal, or parasitical origins.

Radiation pneumonitis is a type of inflammatory response of the lung tissue in response to radiation insult. Indeed, at the cellular level, radiation pneumonitis is characterized by lymphocytic alveolitis, a result of inflammatory infiltrates of mononuclear cells from the vascular compartment into the alveolar spaces. As expected at sites of inflammation, an active interaction between cellular and humoral factors are involved including immune cells, parenchymal cells, macrophages, chemokines, adhesion molecules, lymphocytes, inflammatory cytokines and fibrotic cytokines. Research in radiation pulmonary injury has supported involvement of inflammatory cytokines, chemokines, and fibrotic cytokines. Although investigation of adhesion molecules in radiation lung injury is still underway, these molecules are expected to be involved to serve as prerequisites for leukocyte adhesion to endothelial cells of blood vessels and consequently for transmigration to tissues at sites of inflammation. At the time of clinical symptoms, radiographic infiltrates are often observed in lung volumes, which generally conform to the radiation treatment ports on chest radiographs. The alveolar spaces are filled with patchy infiltrates on chest CT scans and the patients often experience worsening dyspnea. These mononuclear infiltrates may be cleared from alveolar spaces rapidly in response to steroids, likely due to rapid apoptosis of lymphocytes by steroids, and patients often experience marked improvement of dyspnea. With longer follow-up, almost all patients develop radiographic evidences of lung fibrosis.

While current fast-developing new techniques have significantly improved radiotherapeutic gains, radiation-related normal tissue damage still remains unavoidable especially when aiming for optimal tumor control. Normal tissue tolerance, in particular, soft tissue fibrosis, is one of the major dose-limiting factors influencing radiation therapy. It has been reported that radiation-induced soft tissue damage is expected in ten percent of patients when radiation dose is optimized to maximum tumor control. Therefore, a better understanding of the molecular basis of radiation-induced normal damage could provide an effective means for the prevention, or even reversal of radiation-related complications in the clinical radiotherapy. Furthermore, due to the unsatisfactory outcomes of present combination of radiotherapy and chemotherapy, especially with multiple-areas and prolong schedule procedure, much emphasis also are needed to placed on developing better and less side-effects treatment procedure for normal tissue protection.

### SUMMARY OF THE INVENTION

We have discovered that IL-1 is a major contributor to acute and late radiation complications to the bone marrow, bowel, and lungs and soft tissues. We have shown that humans that have high circulating levels of IL-1 before any radiation is delivered develop radiation pneumonitis. In addition, we have found that the absence of IL-1 alpha results in a low propensity for the development of fibrosis following radiation. However, we have also discovered that the elevation of IL-1 persists or rises at later times after radiation.

We have further found that blocking IL function with circulating proteins or drugs is a useful method for the prevention of toxicity to normal tissue and is ethicacious after radiation for the prevention of the progression of toxicity over time.

As a result, the present invention provides for the prevention of and therapy for radiation pneumonitis, dermatitis, soft tissue fibrosis and central nervous system toxicity in patients undergoing therapeutic radiation. In addition, it provides for pre-treatment of those responding to nuclear bio terrorism or other nuclear or radiological accidents. Thus, with the present invention, subjects may be treated in order to prevent toxicity from nuclear bio terrorism or other nuclear or radiological accidents. More particularly, we have discovered a method for profallactically treating radiation toxicity in normal tissue of a subject comprising administering an anti-radiation toxicity effective amount of a cytokine blocking agent through the subject.

More specifically, we have discovered a method for profallactically treating radiation pneumonitis, dermatitis, soft tissue fibrosis or central nervous system toxicity in a subject comprising administering an anti-radiation pneumonitis, dermatitis, soft tissue fibrosis or central nervous system toxicity effective amount of a cytokine blocking agent to the subject.

We have further discovered that the administration of an anti-radiation induced soft tissue effective amount of a COX-2 enzyme inhibitor significantly reduces the amount of tissue damage due to radiation.

We have investigated the role of specific COX-2 inhibitors (Celebrex) in radiation induced soft tissue damage, and explored the relationship between chemokine and its receptor mRNA expression and radiation-induced skin damage in mammary tumor-bearing mice. Here we report that 50 mg/kg Celebrex, given daily with gavage for 15 doses in three weeks, significantly reduced single dose of radiation (60 Gy) induced normal skin damage in MCa-35 mammary tumor-bearing mice. Decreased skin damages are associated with the reduction of the radiation-induced chemokines, Rantes, MCP1, and their related receptor mRNA expression in skin, but not in tumor tissues.

### BRIEF DESCRIPTION OF THE DRAWINGS

FIG. 1 is a graph showing the time scale of the currents of pneumonitis at various time points after radiation;

FIG. 2 is a serious of graphs showing the absolute cytokine level and relative cytokine changes between groups with and without radiation pneumonitis.

FIG. 3 shows the results of circulatory cytokine changes of several cytokines;

FIG. 4 shows plasma levels of Monocyte Chemotactic Protein 1;

FIG. 5 depicts typical changes in gross appearance after radiation of skin;

FIG. 6 Shows a histological changes at various times after radiation;

FIG. 7 graphically depicts the basil levels of IL-B mRNA in mouse skin

FIG. 8 graphically depicts the basil levels of IL-B mRNA in mouse skin

FIG. 9 depicts the circulating IL-1β tissue mRNA expression;

FIG. 10 depicts IL-1α mRNA expression in muscle;

FIG. 11 depicts the effects of radiation on IL-1 Ra mRNA in muscle;

FIG. 12 depicts skin lesions in mice after 20 days of radiation

FIG. 13 depicts inflammation and cellular component infiltration in the dermis in Celebrex treated mice

FIG. 14 summarizes the effects of Celebrex on radiation-induced mRNA expression of chemokines

FIG. 15 depicts the infiltration of inflammatory cells in the derma of Celebrex-treated mice.

### DETAILED DESCRIPTION OF THE INVENTION

### EXAMPLE 1

Materials and Methods: Prospective blood sampling, scoring of respiratory symptoms, and chest imaging were conducted for patients receiving thoracic radiation for malignancy. Serial plasma specimens were analyzed for circulating cytokine changes before, during radiation, and up to 12 weeks post-radiation. Radiation pneumonitis was diagnosed using NCI common Toxicity Criteria. Cytokine analysis was assayed for interleukin a (IL-1α), interleukin 6 (IL-6), Monocyte Chemotactic Protein 1 (MCP-1), E-Selectin, L-Selectin, Transforming Growth Factor β1 (TGF-β1), and Basic Fibroblast Growth Factor (bFGF) using Enzyme Linked Immmunosorbant Assay (ELISA).

Methods

Patient Characteristics

Patients who were to receive thoracic radiation for malignancy were eligible. Blood, thoracic imaging, and respiratory symptom scoring were collected prospectively. Twenty-four patients had follow-up longer than 12 months and their characteristics are shown in Table 1.

TABLE 1
Patient Characteristics

| | Pneumonitis (NCI CTC Grade) | | | |
|---|---|---|---|---|
| | | | Grade 1 | |
| | | | (Radio- | Grade 2 |
| | | | graphic | (Sympto- |
| | All | No | Infiltrates | matic |
| | Patients | Pneumonitis | Only) | Pneumonitis) |
| No. of Patients | 24 | 5 (20.8%) | 6 (25%) | 13 (54%) |
| Age: | 63 (40-80) | | | |
| Median (range) | | | | |
| Sex (M:F) | 11:13 | 2:3 | 3:3 | 6:7 |
| Race (W:H) | 23:01 | 5:0 | 6:0 | 12:1 |
| Histology | | | | |
| Squamous cell | 5 (20.8%) | 2 | 1 | 2 |
| Andenocarcinoma | 11 (45.8%) | 1 | 3 | 7 |
| Andenosquamous | 1 (4.2%) | 0 | 0 | 1 |
| Non-small nos | 3 (12.5%) | 1 | 2 | 0 |
| Small cell | 3 (12.5%) | 1 | 0 | 2 |
| Thymoma | 1 (4.2%) | 0 | 0 | 1 |
| Total | 24 | 5 | 6 | 13 |
| Tumor present | | | | |
| Yes | 21 (87.5%) | 5 | 3 | 13 |
| No | 3 (12.5%) | 0 | 3 | 0 |
| Clinical stage | | | | |
| I | 3 (12.5%) | 0 | 2 | 1 |
| II | 0 | | | |
| III | 15 (62.5%) | 2 | 4 | 9 |
| IV | 3 (12.5%) | 2 | 0 | 1 |
| Limited small cell | 3 (12.5%) | 1 | 0 | 2 |
| Chemotherapy | | | | |
| No | 6 (25%) | 1 | 2 | 3 |
| Yes | 3:15 (75%) | 1:2 | 0:4 | 2:9 |
| (ncocadjuvant: | | | | |
| concurrent) | | | | |

Abbreviations: NCI CTC, National Cancer Institute common toxicity criteria; nos, not otherwise specified.

Clinical and Radiographic Evaluation

History and physical examinations emphasizing the respiratory symptoms were performed periodically. Clinical evaluation for pulmonary symptoms was evaluated and graded using the LENT/SOMA scoring system for the lung. This system includes the RTOG treatment side effect scoring of subjective clinical symptoms, and an objective assessment of serial chest X-rays and CT scan changes. Pneumonitis grading was also defined according to NCI Common Toxicity Criteria.

Circulating Cytokinie Analysis

Plasma samples were collected before therapy and weekly, during therapy. Specimens were collected in sodium heparin as well as EDTA up to 12 weeks post-therapy. Platelet-free plasma was produced by centrifugation at 1200 rpm at 0° C. for 10 minutes. The plasma was stored in aliquots at −20° C. Heparinized plasma was used for the analysis of most cytokines and EDTA plasma was used for the analysis of bFGF. Cytokines were analyzed using Enzyme-Linked Immunosorbent Assay (ELISA). The methodology of ELISA analysis was according to manufacturers' instructions as previously described.

Twenty-four patients had clinical follow-up longer than 12 months after radiation. Thirteen developed symptomatic pneumonitis (NCI grade 2). The peak incidence of symptoms was between 6- and 13 weeks post radiotherapy. Six patients had only radiographic infiltrates. (NCI grade 1). Five patients did not have clinical or radiographic pneumonitis. Both IL-1α and IL-6 levels were significantly higher before, during, and after radiation for those who developed pneumonitis. The pattern of changes of MCP-1, E-Selectin, L-Selectin, TGF-β1, and bFGF varied but none of these cytokines correlated with radiation pneumonitis.

Analysis of a panel of circulatory cytokines with different putative function in radiation pulmonary injury showed that pre-treatment IΛ-1α and IL-6, as well as mid and post-treatment levels were significantly higher for patients who subsequently developed radiation pneumonitis.

Radiation Pneumonitis

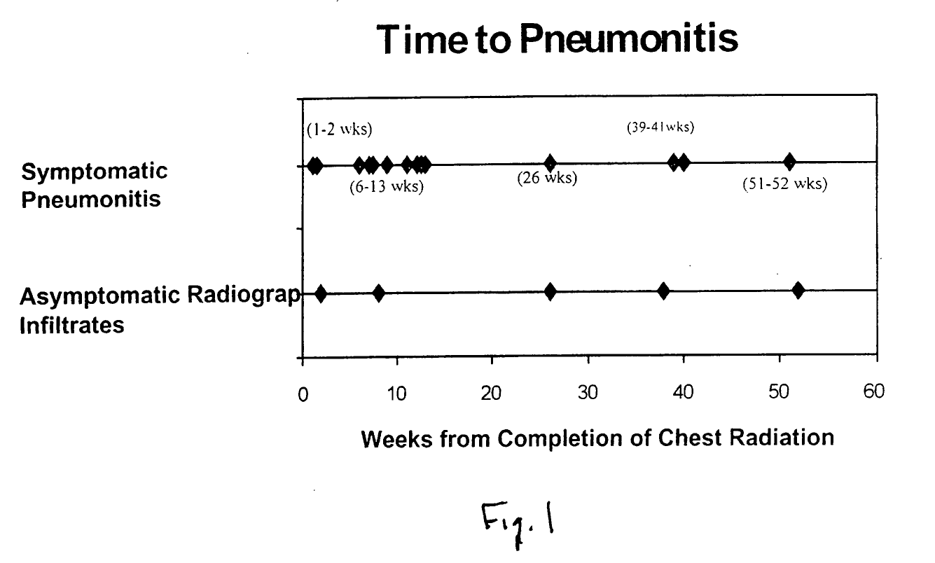

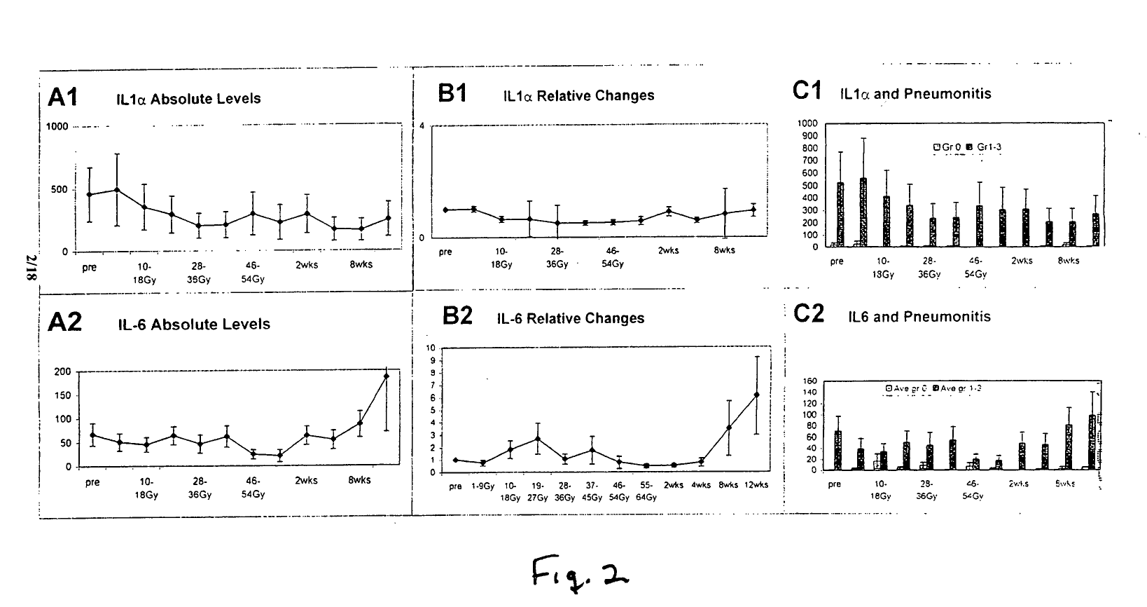

Symptomatic radiation pneumonitis is characterized by an annoying cough that is either non-productive or with clear sputum. This period is generally accompanied by markedly worsening dyspnea in an otherwise healthy appearing individual. Generally there are also radiographic infiltrates on chest x-ray and CT scan that usually conforms to radiation ports. The individual in general is afebrile or has a low-grade temperature, and is without an increase of blood neutrophil counts. Clinical symptoms are rapidly relieved with low dose steroid treatment. Of the 24 patients with follow-up longer than 12 months, 13 developed clinical symptoms consistent with radiation pneumonitis (NCI grade 2 pneumonitis). Six had radiographic infiltrates only, without clinical symptoms (NCI grade 1). Five did not have either radiographic infiltrates or clinical symptoms. The timescale of occurrence of pneumonitis is shown in FIG. 1. As demonstrated in FIG. 1, asymptomatic infiltrates occurred at random time points after radiation, while symptomatic pneumonitis occurred most commonly between 6 weeks and 13 weeks after completion of radiation. For all symptomatic pneumonitis, the first episodes all occurred within 6 months post-radiotherapy. The outliers beyond 6 months in FIG. 1 were those with recurrence of symptomatic pneumonitis. In all these cases, however, the first symptoms had occurred within 6 months after therapy.

Pro-inflammatory Cytokinies Markers: IL-1α and IL-6

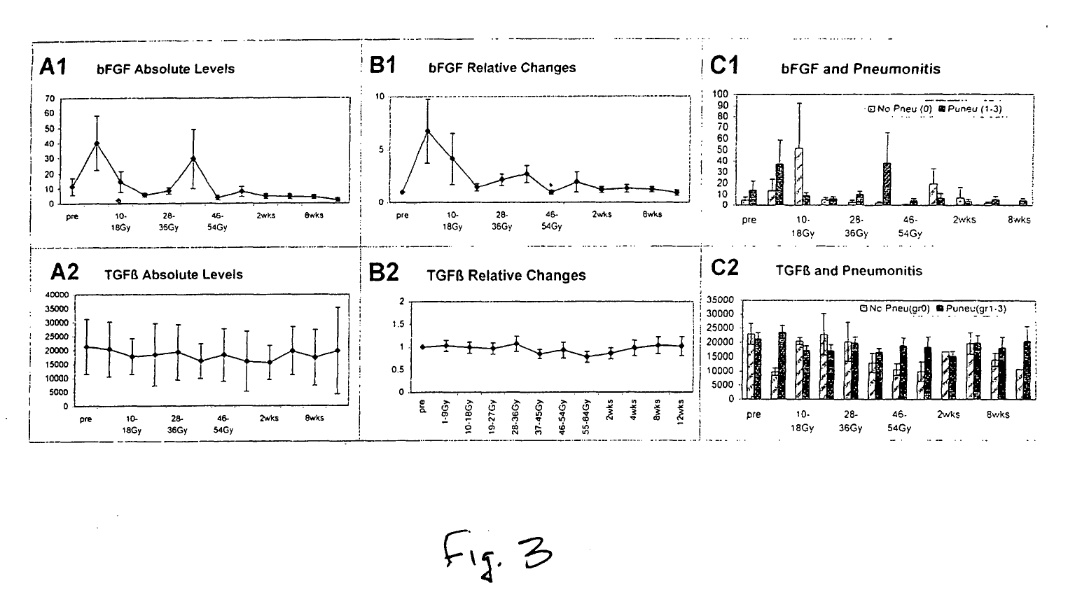

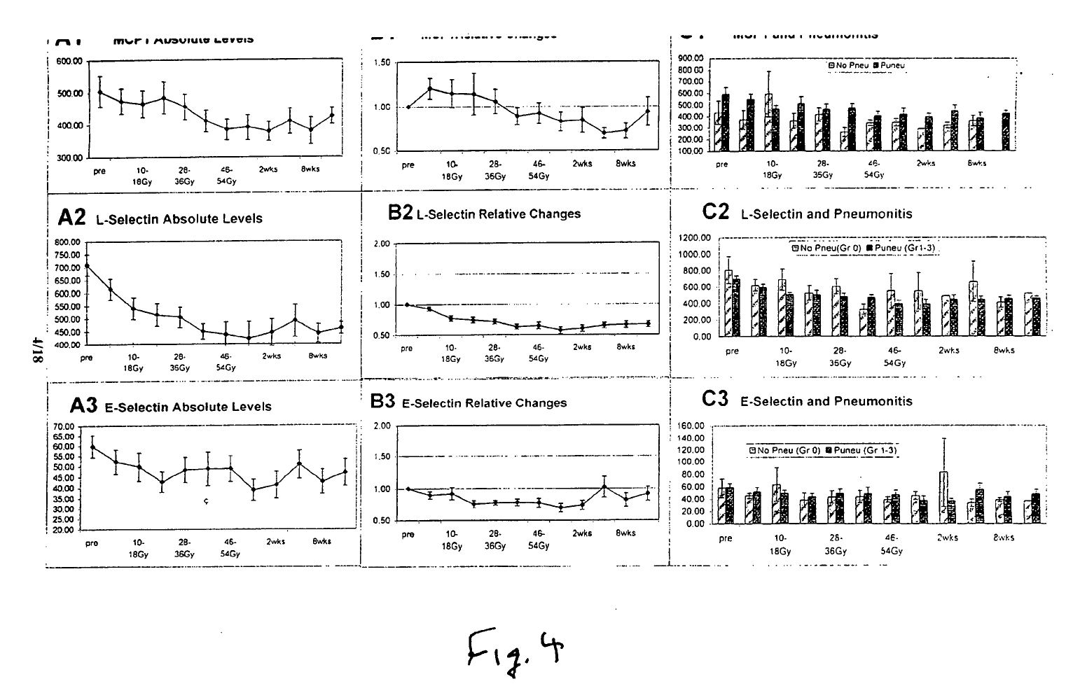

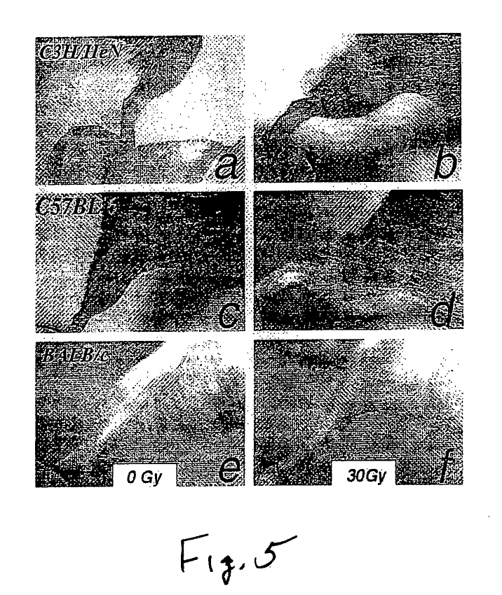

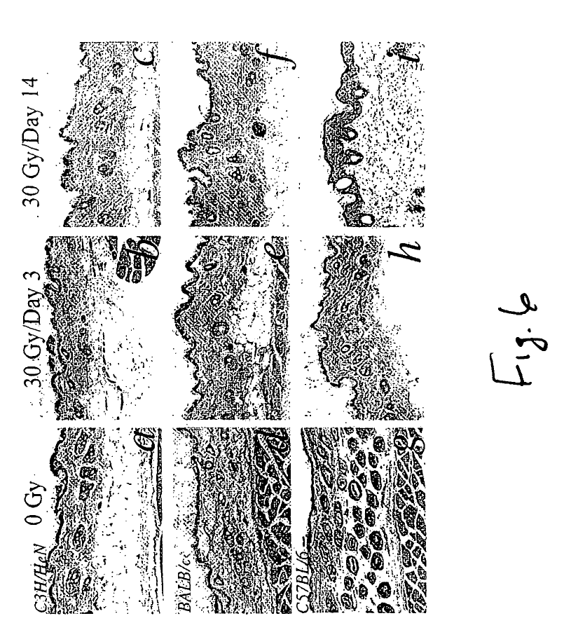

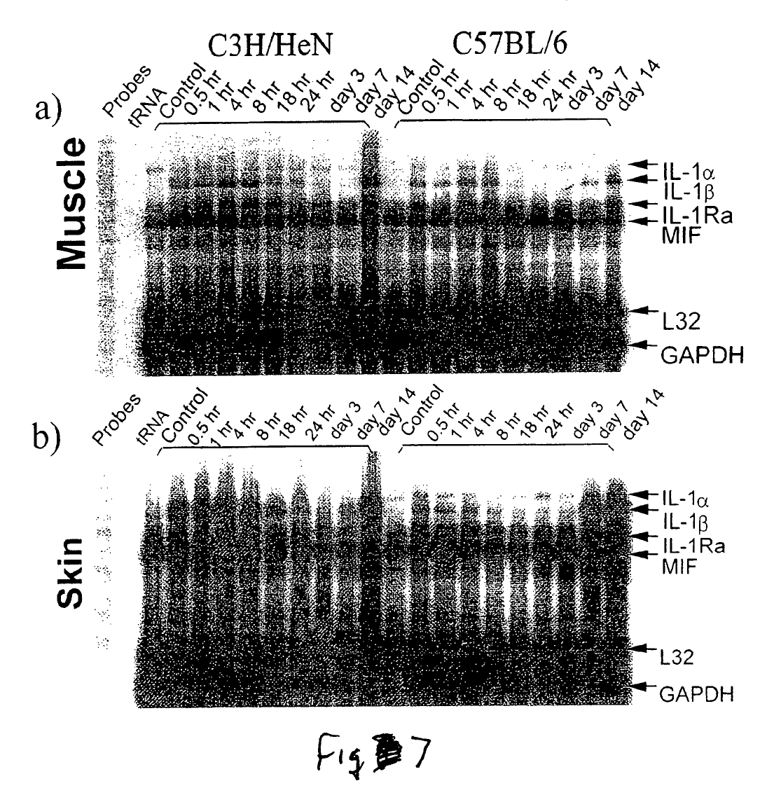

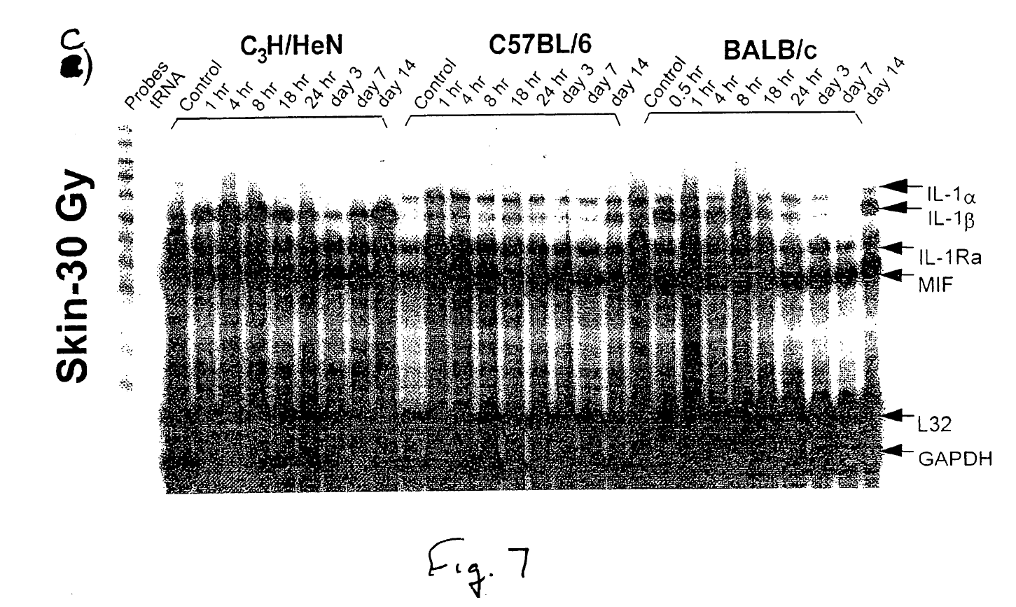

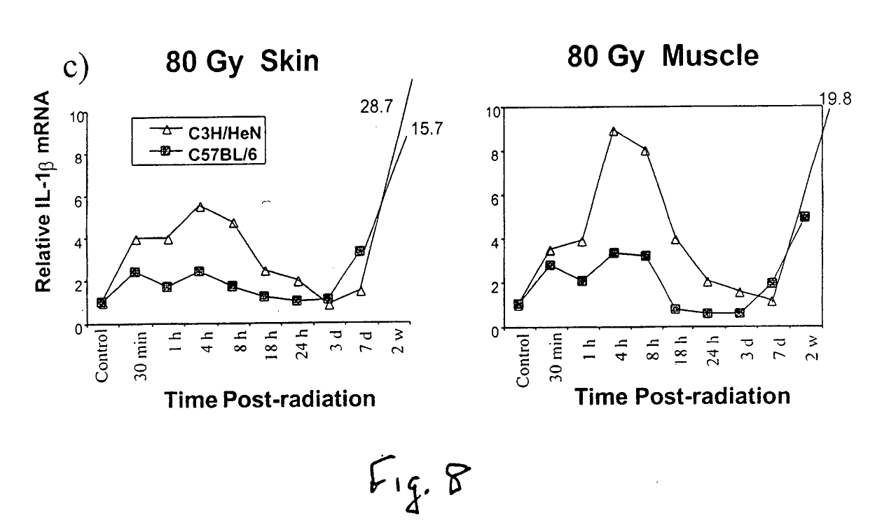

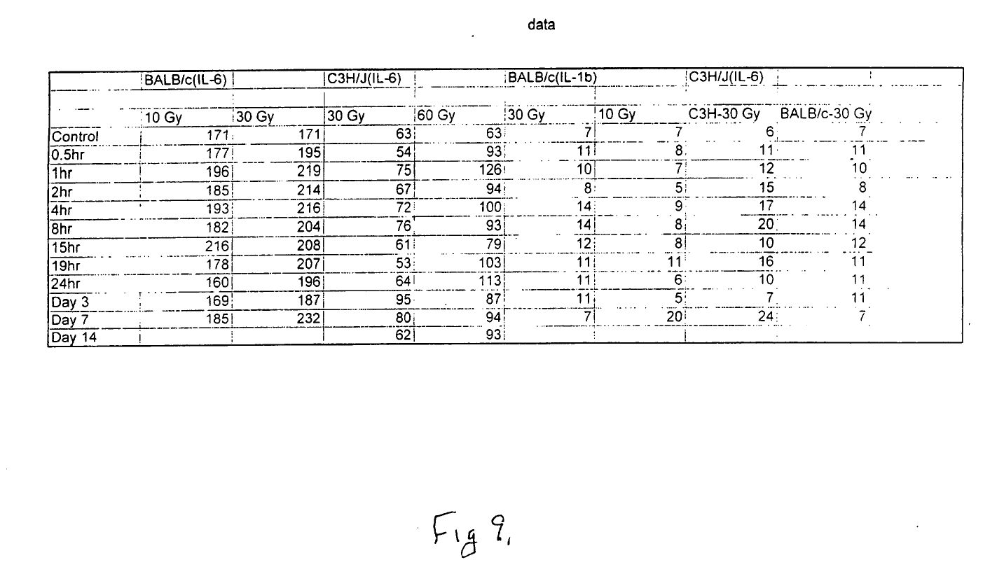

We analyzed pro-inflammatory cytokine IL-1α, and IL-6 levels before radiation treatment, weekly during treatment, and up to 12 weeks following radiation. FIG. 2 shows the absolute cytokine level (in pg/ml) (A1 for IL-1α, and A2 for IL-6) and the relative cytokine changes normalized to individual pre-treatment value (B1 for IL-1α, and B2 for IL-6), as well as the comparison of absolute values between the groups with and without radiation pneumonitis (C1 for IL-1α, and C2 for IL-6). The data showed a very wide range of individual circulatory IL-1α levels (A1), but a relative lack of changes with radiation treatment (B1). In contrast to IL-1α, IL-6 levels were not as variable among individuals (A2), but they fluctuated somewhat with radiation. Of note, after completion of radiation treatments, there is a trend toward an increase of IL-6 in both absolute levels and relative changes (A2 and B2, p=0.065). Both IL-1α and IL-6 absolute levels were significantly higher before radiation, at multiple time points during radiation, and after radiation (C1, and C2, p<0.05) in patients who subsequently developed radiation pneumonitis.

Pro-fibrotic Cytokine Markers: bFGF and TGF-β1

FIG. 3 demonstrates results of circulatory cytokine changes of fibrotic cytokines bFGF and TGF-β1. Basic FGF levels fluctuated during treatments and showed no correlation with pneumonitis (A1, B1, and C1). In contrast, there were many individual variations of circulatory TGF-β1 (A1), but there was much lesser degree of relative changes during radiation and after radiation up to 12 weeks post-therapy. Similar to bFGF, circulating TGF-β1 did not show an appreciable difference between the group with and the group without pneumonitis (C2).

Chemokine and Adhesion Molecule Markers: MCP-1, L-Selectin, and E-Selectin

Plasma levels of MCP-1 (Monocyte Chemotactic Protein 1), L-Selectin, and S-Selectin (FIG. 4) were also measured. FIG. 4A demonstrates the absolute levels of MCP-1 (A1), relative changes of MCP-1 (B1), and the comparison of the groups with and without pneumonitis (C1). Our data showed a decline of MCP-1 levels during the last week of radiation and up to 8 weeks after radiation (p<0.04). Data on L-Selectin demonstrated a marked and significant decline of the circulatory levels of L-Selectin (A2, p<0.01) and the relative changes (B2, p<0.01), and a lack of difference between the pneumonitis group and the non-pneumonitis group. There was some decline of circulatory MCP 1 near the end of radiation up to 8 weeks after treatments. Data on E-Selectin is similar to L-Selectin in that there was some decline of levels near the end of radiation and after radiation (p<0.03) as well as a decrease of relative changes through most time points of the period investigated (p<0.01). There also was not a significant difference between the pneumonitis group and the non-pneumonitis group.

Radiation pneumonitis and fibrosis can be regarded as the consequences of a wound-healing inflammatory reaction to radiation damage of lung tissues. Research in immunological regulation of inflammation has revealed the complex interaction between local tissues and immune cells mediated through chemokines, adhesion molecules, inflammatory cytokine, and fibrotic cytokines.

Inflammatory Cytokines and Radiation Pneumonitis

We have shown that lung radiation is associated with a temporal expression of IL-1α, TGF-β1, collagen I, collagen III, and collagen IV gene expression in fibrosis-prone mice (C57BL/6). Among the panel of cytokines potentially involved in the inflammatory response to radiation lung injury, IL-1α and IL-6 were the only two cytokines that correlated significantly with radiation pneumonitis (FIG. 4). In addition, pre-treatment levels of both IL-1α and IL-6 were significantly higher in patients who subsequently developed pneumonitis, supporting their role as predictors of radiation pneumonitis. Our data showed some differences between IL-1α and IL-6, however, in that when normalized to individual pre-treatment levels, IL-1α remains relatively constant during treatment course, but there is a trend toward elevation of IL-6 at 8 to 12 weeks post-radiation.

The rise of IL-6 after completion of radiation was observed. It coincided with the period of clinical symptomatic pneumonitis and this deserves further investigation (FIG. 1). Both IL-6 and IL-1α are important immunoregulatory moieties. Although both are inflammatory cytokines, they differ somewhat in origin of cells and in some functional aspects. Both cytokines mediate fever and regulate inflammation and fibrotic response through immune cells. The source of IL-1 is primarily from monocytes as well as alveolar macrophages. IL-6 is synthesized by a variety of cells in the lung parenchyma, including the alveolar macrophages, type IT pneumocytes, T lymphocytes, and lung fibroblasts. In the in vitro system, when alveolar macrophages were exposed to clinically relevant dose of radiation (2 Gy), it was found that both IL-1α and IL-1β were released in increased amounts. It has been shown that IL-1 stimulates human lung fibroblast in the production of IL-6 and stabilizes IL-6 messenger RNA production. In patients with higher pre-treatment levels of IL-1α, IL-1α may regulate a subsequent increase of IL-6 after radiation, was observed (FIG. 2).

Pro-fibrotic Cytokines and Radiation Pneumonitis

Pro-fibrotic cytokines participate in radiation lung injury, especially during the development of lung fibrosis phase, which generally starts at 4 to 6 months after treatment and continues without a clear end point. Lung fibrosis is equivalent to the scar after the initial inflammatory phase of lung reaction to radiation injury. Although radiographic fibrosis in general is not observed until 4 to 6 months after completion of radiation, it has been reported that circulatory TGF-β1 changes may serve as an early predictor for radiation pneumonitis and its expression increases with radiation in animal research models. Two pro-fibrotic cytokines, bFGF and TGF-β1, and their changes in the association to radiation pneumonitis (FIG. 3) was investigated. As the incidence of radiation pneumonitis peaks at 6 to 13 weeks in our cohort of patients, we analyzed our data up to 12 weeks and did not find an association in predicting radiation pneumonitis with either bFGF or TGF-β1. This finding may be attributed to the patient population and sample size differences. Since cytokines are relatively fragile molecules, technical differences in specimen collection, processing, and laboratory assays may also result in the differences in laboratory measurements.

We have discussed that circulatory measure of IL-1α and IL-6 turned are significantly associated with radiation pneumonitis. Thus, patients with higher baseline levels of inflammatory cytokines are more vulnerable to radiation lung injury.

Figure Legends:

FIG. 1. Twenty-four patients were followed prospectively for clinical symptoms of radiation pneumonitis and radiographic changes. The scattered plot demonstrates the time of either symptomatic pneumonitis (top line) or only radiographic infiltrates without symptoms (bottom line). Data showed that symptomatic pneumonitis was diagnosed primarily between 6 weeks and 13 weeks after completion of radiation with rare outliers occurring prior to 6 weeks and between 6 months to 9 months.

FIG. 2. IL-1α absolute levels (A1), relative changes normalized to individual pretreatment levels (B1), and the comparison of levels between patients with grade 1 to 3 pneumonitis (solid bar) and no pneumonitis (hatched bar) are presented for pre-treatment baseline level, weekly during radiation, and up to 12 weeks after radiation. FIG. 2 A2, B2, and C2 demonstrate the IL-6 absolute levels, and relative changes and the comparison between the two groups of patients, respectively.

FIG. 3. Basic FGF a absolute levels (A1), relative changes normalized to individual pretreatment levels (B1), and the comparison of levels between patients with grade 1 to 3 pneumonitis (solid bar) and no pneumonitis (hatched bar) are presented for pre-treatment baseline level, weekly during radiation and up to 12 weeks after radiation. FIG. 3 A2, B2, and C2 demonstrate the TGF-β1 absolute levels, relative changes, and the comparison between the two groups of patients, respectively.

FIG. 4. Basic MCP1 absolute levels (A1), relative changes normalized to individual pretreatment levels (B1), and the comparison of levels between patients with grade 1 to 3 pneumonitis (solid bar) and no pneumonitis (hatched bar) are presented for pre-treatment baseline level, weekly during radiation and up to 12 weeks after radiation. FIG. 4 A2, B2, and C2 demonstrate the L-Selectin absolute levels, relative changes, and the comparison between the two groups of patients, respectively. In FIG. 4, A3, B3, and C3 demonstrate the E-Selectin absolute levels, relative changes, and the comparison between the two groups of patients, respectively.

### EXAMPLE 2

Materials and Methods

Mice Strains and Radiation Treatment

Six to 7 week-old female C3H/HeN, BALB/c and C57BL/6 mice were used (Jackson Laboratories, Bar Harbor, Me.). The right hind leg (10 mice per group) was given 10, 20, 30, 40, 60, or 80 Gy in a single radiation dose with a Shephered Irradiator, a 6000 Ci Cs source, together with collimating equipment. The left, non-irradiated hind leg was used as the non-irradiated control. Mice were sacrificed at different time points after radiation (0.5, 1, 2, 4, 8, 12hrs, day 1, day 7, and day 14). At least 10 mice were used at each time point. Tissues from 3 mice were used for histology, and the remaining animals were used for mRNA analysis. Skin and muscle tissues from control and irradiated legs were dissected, and total RNA was isolated. Guidelines for the humane treatment of animals were followed as approved by the University of Rochester Committee on Animal Resources.

Tumor Tissue RNA Isolation and RNase Protection Assays

Skin and muscle tissues from each treatment group (7-10 mice) were pooled and total RNA was isolated by pulverizing the frozen tissue and dissolving it in TRI Reagent (Molecular Research Center, Ohio) according to the manufacturer's specifications. To determine the integrity of isolated RNA, 2 μg of RNA from each sample was fractionated on a formaldehyde gel and visualized by staining in ethidium bromide. RNase protection was performed using established multi-probes template sets (PharMingen, SanDiago, Calif.) as described previously. The interleukin (IL) sets include: IL-1α:, IL-1β, IL-1Rα, IL-6, IL-10 and IL-12. Two internal controls, L32 and GAPDH, were used as loading controls. The cocktail constructs were used to prepare P-UTP labeled antisense cRNA probes using the PharMingen in vitro transcription kits (PharMingen, SanDiago, Calif.). Probes were hybridized with 30 μg of total RNA at 50° C. for 16 hr. RNase A (1 mg/ml) and RNase T1 (2000 U/ml) were then added to digest single-stranded RNA. After digestion, the RNA was precipitated and resuspended in gel loading buffer, heated at 95° C. for 5 min, and run in 7% denaturing polyacrylamide gel (National Diagnostics, Ga.). The gel was run for 2-3 hr at 60v, dried on Whatman filter paper, and placed on a phosphorimager screen for quantitative analysis using a Cyclone Phosphorimager device (HP Company, Conn.). Area integration of each mRNA-protected fragment was normalized against the protected internal control band (GAPDH) in the corresponding lane to calculate the ratio of targeted/GAPDH mRNA. In order to compare the basal levels with radiation-induced levels for each interleukine mRNA tested, relative mRNA levels (folds) were plotted. Some gels are shown with over-exposure of the control lanes to highlight differences in IL-1α/β expression.

Blood Cytokines Assays (ELISA)

Blood samples were collected from 3 mice strains at various time points after radiation. After centrifugation for 30 minutes at 4° C., plasmas were aliquated and stored at −70° C. until analysis. Immunoenzymetric assays for murine IL-1β (Endogen Inc, Cambridge, Mass.) were performed according to the manufacturer's instructions. A standard curve with cytokine-positive control was run in each assay and the lower limit of detection was determined to be 3.5 pg/ml. Most of non-irradiated mice had circulating IL-1β protein levels near the limit of detection.

In Situ Hybridization

Localization of the IL-1β gene in soft tissue was determined by in situ localization and was performed as previously published. Briefly, leg tissues were fixed in 10% formalin and 2% paraformadhyde by cutting the whole leg into 3-5 pieces. Tissue sections were then placed on specially prepared slides (acid washed and T3-aminopropyl trietlioxysilane coated) and were deparaffinized and rebydrated. Proteinase K-digested sections were hybridized with appropriate amounts of IL-1β riboprobe. Sections to be examined were hybridized with anti-sense RNA under conditions of probe excess, and, after washing, they were prepared for autoradiography using NBTII emulsion (Kodak, Rochester, N.Y.). After autoradiography and staining, the slides were analyzed by bright and dark field microscopy. Backgrounds for these studies were determined using the sense stand RNA probe. As positive controls for hybridization, some sections were hybridized with constitutively expressed mRNA (GAPDH) and were analyzed for cell specific expression of the molecule of interest. Cell types and locations of IL-1β over-expression were identified histologically.

Statistical Analysis

Cytokine mRNA expression levels from skin and muscle in non-irradiated versus irradiated tissues were compared using the unpaired Student's t-test, or Mann-Whitney Rank Sum test as appropriate. Differences were considered significant for p<0.05.

Results

Pathological Observation

At early time points after irradiation of the skin, the gross appearance was only mildly different from one strain of mice to another. FIG. 5 shows typical changes seen after 30 Gy. During this acute process, which occurs over the 14 days following limb irradiation, the C3H/HeN mice (least fibrosis sensitive strain) had some hair loss and leg swelling (FIG. 5b). The BALB/c mice, which have intermediate fibrosis sensitivity, had the most edema and hair loss (FIG. 5f), while the fibrosis sensitive C57BL/6 mice had only a thinning of the fur with minimal edema over the first 14 days (FIG. 5d). Local hair loss was noted during the first 14 days in all 3 mice strains, in a dose dependent manner. Ulceration was seen only in the high dose groups (60 and 80 Gy) at 14 days, and it was less common in the C57BL/6 mice. However, the acute inflammation that occurred in these animals over the 2 week observation period did not correspond to the degree of fibrosis that is expected 2 months after therapy.

The histological changes mirrored the clinical examinations, with some qualitative differences. As an example, 30 Gy irradiated tissues at various times after treatment are shown in FIG. 6. C3H/HeN and BALB/c mice had lower basal densities of hair follicles compared to C57BL/6 mice (FIG. 6a, d, and g). Three days after 30 Gy irradiation, all 3 strains had similar follicle densities; however, the sub-epidermal matrix accumulation was more pronounced in BALB/c and C3H/HeN mice (FIG. 6c and f). At 14 days, inflammatory cells and fibroblasts in the dermis were more pronounced in C57BL/6 mice (FIG. 6).

Expression of IL-1β mRNA

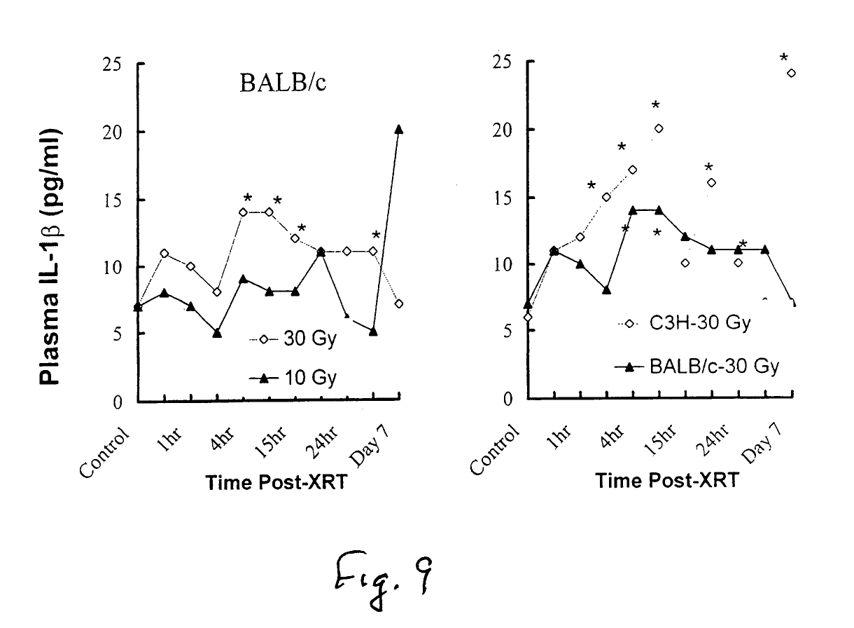

In order to determine the molecular correlation of radiation-induced soft tissue damage, we examined mRNA expression of interleukins, IL-1α, IL-1β, and IL-1Ra by RNase protection assay in skin and muscle tissues from the 3 mice strains before and after different doses of radiation. As shown in FIG. 7 and FIG. 8, and summarized in Table 2, all 3 mice strains had detectable basal levels of IL-β mRNA in their skin (C3H/HeN mice had the highest), but only very low levels of IL-1β mRNA in muscle. Skin IL-1β mRNA expression was substantially increased within a few hours following 30 Gy leg irradiation (FIG. 3). There were two phases of IL-1β mRNA elevations: first from 0.5 to 4 hrs and then from 7-14 days following irradiation (FIG. 7a, b, and c). C3H/HeN and BALB/c mice had similar patterns of IL-β mRNA expression after irradiation (FIG. 7). In contrast, the fibrosis-sensitive C57BL/6 mice had little if any IL-1β mRNA induction. Neither of the bimodal peaks were seen in irradiated muscle of C57BL/6 mice. Elevation of skin and muscle IL-1β mRNA in C3H/HeN and BALB/c mice was radiation dose-dependent. Very high dose (80 Gy) radiation significantly increased mRNA expression of skin and muscle IL-β (6 and 9 fold, respectively) in C3H/HeN mice at 4 hrs (FIG. 8a, b, and c). After 14 days, the 80 Gy dose significantly increased IL-1β mRNA levels in both C57BL/6 and C3H/HeN mice, which were over 15-fold higher than those of non-irradiated controls. Local leg radiation not only increased local tissue IL-1β mRNA expression, but it also increased circulating IL-1β measured by ELISA. Circulating IL-1β was associated with tissue mRNA expression with bimodal elevations at 4-8 hr and again at 7 to 14 days in both BALB/c and C3H/HeN mice (FIG. 9b). There appeared to be a dose response, with higher local radiation doses leading to chronically higher circulating IL-1β levels. In order to define the cell types producing IL-1β mRNA, in situ hybridization was performed on irradiated soft tissues. Increased IL-1β mRNA was mainly localized in keratinocytes and stroma cells in the dermis of non-irradiated skin.

Expression of IL-1α mRNA

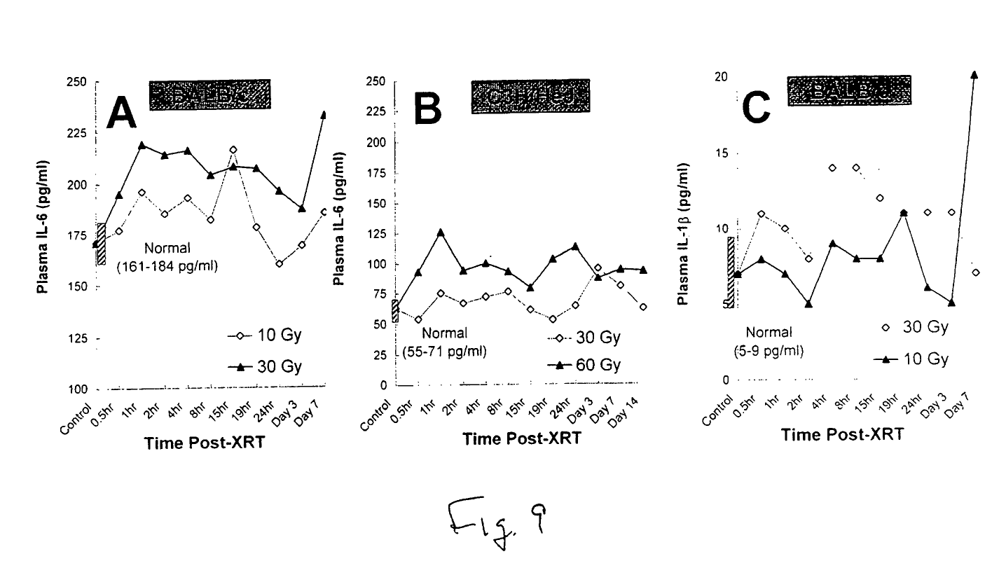

As shown in FIG. 7 and Table 2, undetectable or very low levels of IL-1α mRNA were measured in the skin of C3H/HeN mice. A 2 to 6 fold induction of skin IL-1α mRNA was detected in both C57BL/6 (FIG. 7a) and BALB/c mice after high doses of radiation (FIG. 10a and 10b). This increased skin IL-1α mRNA expression was radiation dose-dependent, progressed with time, and was minimal at the sub-fibrogenic radiation doses (≦30Gy). Radiation did not appear to induce IL-1α mRNA expression in muscle of any of the 3 mice strains (FIG. 10c).

Expression of IL-1Ra mRNA

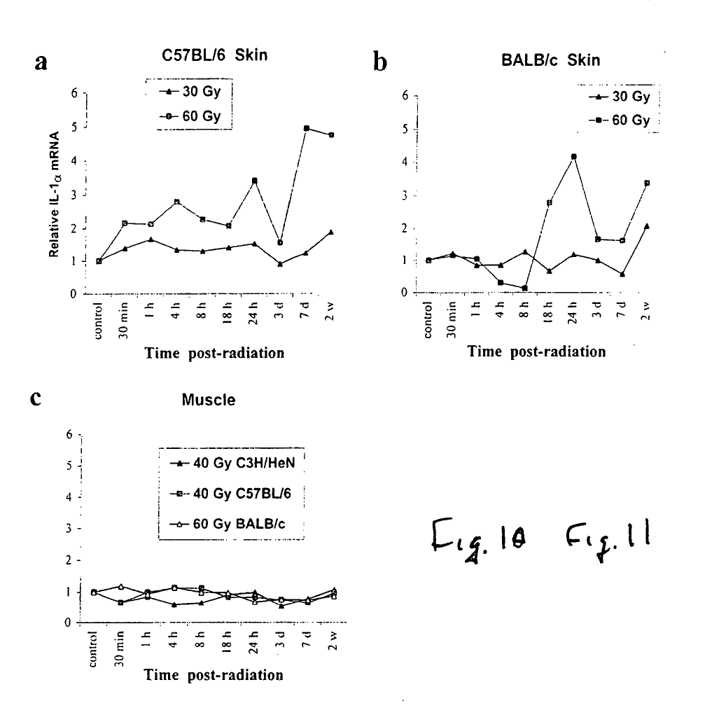

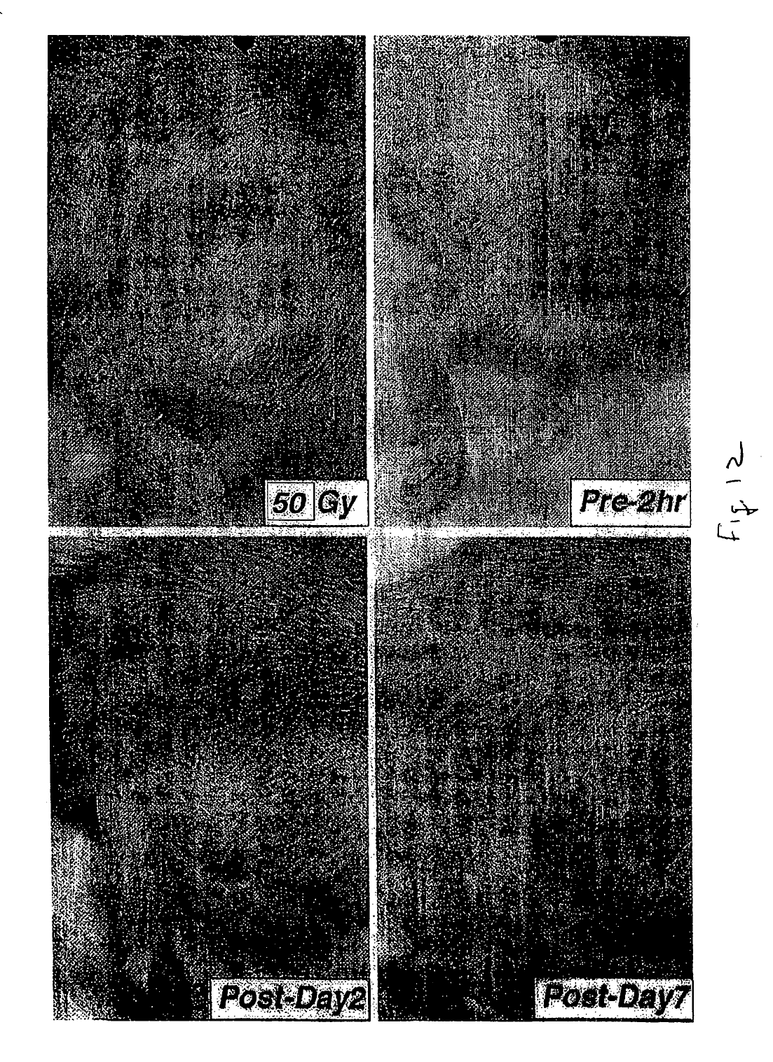

Like IL-1β mRNA, IL-1Ra mRNA was highly expressed in skin tissue, and no substantial difference in the basal levels of IL-1Ra mRNA was seen among the three strains (FIG. 11). Skin IL-1Ra, however, was dramatically induced by radiation in C57BL/6 mice, but not in C3H/HeN or BALB/c mice. Induction of IL-1Ra mRNA in C57BU6 mice was radiation dose dependent. The effects of radiation on IL-1Ra mRNA expression in muscle of any strain was minimal (FIG. 11d).

Discusssion

Murine models were used to simulate the situation that occurs in human skin after irradiation. This enabled us to examine the molecular characteristics of soft tissue fibrosis. Doses that caused little or no fibrosis (<30Gy), as well as highly fibrogenic doses (60-80Gy) were used in the 3 mice strains. We expected that, if radiation-induced cytokine mRNA expression is a causal event, then high doses would induce higher levels of cytokine mRNA, explaining strain variation in fibrosis sensitivity. Two key questions were asked in this study: 1) Is there a difference in basal mRNA expression of certain cytokines in skin or muscle tissues among 3 mouse strains with different fibrosis sensitivities? 2) Does this difference in mRNA expression contribute to the various radiation-related fibrosis responses in the three mouse strains? We demonstrated that: 1) skin tissues express higher levels of several interleukins than muscle tissues, independent of mouse strain. This is consistent with prominent initial fibrosis occurring in the subepidermal regions, with less and later development of fibrosis in muscle tissue. 2) C3H/HeN mice have the lowest predisposition for developing fibrosis and did not express IL-1α mRNA in their skin. The most fibrosis sensitive strain, C57BL/6, had high basal and radiation-induced levels of this cytokine. Muscle, which is more fibrosis resistant than skin, also had lower or undetectable IL-1α expression compared to skin. 3) Radiation induced elevation of IL-1β mRNA was biphasic with an early peak (1 to 4 hr) and another at a later time (3 to 14 day). The first phase was absent in the fibrosis sensitive strain, and it was intermediate in the strain with intermediate fibrosis sensitivity. 4) Cytokine responses in muscle were more blunted, compared to those in skin, and required higher radiation doses. 5) Cytokine responses after local radiation could be large enough to be detected in the circulation. 6) The cells synthesizing the greatest quantities of IL-1β appear to be the keratinocytes and stromal cells of the epidermis and dermis. Taken together we propose that these patterns suggest that brisk IL-1α responses to radiation and high basal IL-1α mRNA levels are associated with a higher risk for late radiation fibrosis. An early pulse of IL-1β expression after irradiation appears to correlate with a lower risk for developing radiation soft tissue fibrosis. The data also provided evidence that circulating levels of cytokines might be a useful marker of local cytokine production following radiation.

It has been demonstrated both experimentally and clinically that high basal levels of fibrogenic cytokines and/or growth factors are related to a higher incidence of radiation- or chemotherapy-induced late tissue damage. Our recent animal studies also suggest that high blood TGF-β levels are associated with a high risk for radiation-induced tissue fibrosis. We measured local and circulating levels of interleukin mRNA in our 3 mice strains with different fibrosis sensitivities because higher basal mRNA levels of these cytokines may also be related to a higher risk of radiation-mediated normal tissue fibrosis. It is apparent from our data that C3H/HeN skin does not have detectable IL-1α mRNA. Low or undetectable skin IL-1α mRNA in C3H/HeN mice, a fibrosis resistance mouse strain, may be responsible for its resistant phenotype. In our radiation-induced lung fibrosis models, similar results were also observed. The correlation of low mRNA levels of skin and lung IL-1α with increased resistance of radiation-induced fibrosis warrants further investigation.

Radiation-induced expression of interleukin mRNA is organ-dependent. All interleukin responses were more pronounced in the skin than in muscle. Inducible levels of each cytokine, however, varied between skin and muscle tissues. For example, radiation induced an elevation of skin IL-1α mRNA, not muscle IL-1α mRNA, in C57BL/6 mice. Our previous data in cultured cell lines (keratinocytes, skin fibroblast, and squamous cell carcinoma cells) also demonstrated that different cell types not only express different levels of each cytokine, but also respond to radiation differently. Our data here may also provide some guidance for clinical radiation therapy. For example, avoidance of cutaneous radiation might prevent cytokine cascades that could result in late tissue fibrosis. This is because soft tissue fibrosis begins in the subepidermis, later extends through the dermis, and eventually involves the superficial and the deeper muscle layers. Clinically, the efficacy of megavoltage radiation is in large part due to the lower epidermal dosimetry. It is an intriguing notion that patients with elevated basal IL-1α mRNA might be treated prophylactically with anti-cytokine therapy to prevent fibrosis.

While radiation-induced alteration of interleukin mRNA in lung and other organs have been reported in several strains of mice, altered mRNA levels of cytokines in soft tissues from different strains of mice have not yet been reported. In this study, we collected and processed RNA samples of three strains in the same RNase protection gel, and we also compared the IL-1 mRNA expression difference between skin and muscle. We found that the patterns of cytokine mRNA expression were consistent with the degree of fibrotic response. In contrast, macroscopic and microscopic acute alterations were weak predictors of fibrosis sensitivity. The lack of correlation between acute reactions and late effects has been studied for decades, and the role that cytokines and growth factors play appears to finally help explain the phenomenon.

Radiation increased IL-1 mRNA expression in two waves, the first at approximately 4 hours after therapy and another 3 to 14 days post-radiation. Examination of corresponding skin tissue morphology at each time point suggested that acute tissue response in preexisting cellular components may be responsible for the first peak of cytokine production. In situ hybridization studies suggest that keratinocytes, endothelial cells, and skin fibroblasts are the source of the early IL-1β mRNA expression. Infiltrating inflammatory cells and activated fibroblasts are probably responsible for the second peak in cytokine mRNA production. Several studies have demonstrated that pulses of IL-1, given within 24 hours of radiation, are radioprotective. Endogenous pulsing of IL-1β in C3H/HeN mice after radiation may therefore partly explain this strain's higher resistance to fibrosis compared to C57BL/6 mice.

In conclusion, we have shown that skin tissues produce more interleukin mRNA compared with muscle tissues. Skin IL-1α and IL-1Ra mRNA are upregulated in C57BL/6 mice, while IL-1β mRNA is increased in C3H/HeN and BALB/c mice within a few hours of local leg radiation. These results show that radiation-induced differential mRNA expression for interleukin and varied basal levels of interleukin mRNA participate in radiation-induced normal tissue damage.

Legends

FIG. 5. Typical gross observation of radiation changes seen in control (a, c and e) and 14 days following 30 Gy irradiation (b, d and f) of the right hind limb in 3 mice strains. Edema was similar in all three strains, and hair loss was similar in C3H/HeN and C57BL/6 mice, with slightly greater hair loss in BALB/c mice (f).

FIG. 6. The characteristic histological observation of progressive pathological changes of radiation fibrosis are shown in panels a through i. Normal mouse skin for C3H/HeN (a), BALB/c (d), and C57BL/6 (g). Note the thin epidermis with underlying papillary dermis, hair follicles containing multiple hairs. Leg muscle is free of significant inflammation. Day 3 (b, e, and h) and day 14 (c, f, and i) after 30 Gy radiation are shown. Early soft tissue reaction includes progressive loss of dermal papilla, reduced hair follicle number, increased empty hair follicles, and a superficial filling of the dermis with matrix and inflammatory cells. There is little inflammation of muscle, and the dermal inflammatory cell infiltrates were grossly similar in all strains.

FIG. 7. IL-1β mRNA expression in irradiated limbs in 3 mice strains by RNase protection assay (a). IL-1β mRNA expression was quantitatively determined using a Cyclone PhosphorImager (HP Co, MI). IL-1β mRNA values are pooled from seven mice per measurement for irradiated skin (b) and muscle (c). Lanes are shown over-exposed to demonstrate the absence of IL-1α in the skin of C3H/HeN mice, and the brisk IL-1β response to radiation in C3H/HeN and BALB/c but not in C57BL/6. The early phase of IL-1β mRNA expression was seen in muscle, while the later increase at 1 to 2 weeks was less evident in muscle. 30 Gray is sufficient to cause a high frequency of severe acute reactions in all strains, but, at 2 months following radiation, 30 Gy is sub-fibrogenic for most C3H/HeN and BALB/c mice.

FIG. 8. Determination of IL-1β mRNA expression in high dose (80 Gy) irradiated limbs from C3H/HeN and C57BL/6 mice by RNase protection assay (a and b). mRNA from seven mice was pooled. 80 Gy radiation induced elevated IL-1β mRNA expression in both skin and muscle tissues. 80 Gy is sufficient to cause substantial fibrosis and acute reaction in all strains.

FIG. 9. Plasma IL-1β levels in C3H/HeN and BALB/c mice after limb irradiation. Circulating levels of IL-1β in platelet depleted plasma were significantly increased after 30 Gy radiation in BALB/c mice (left). The difference from baseline was not significant at any time after 10 Gy, which is a sub-fibrogenic dose. In a separate experiment (right), 30 Gy radiation significantly increased blood IL-1β in both C3H/HeN and BALB/c mice. The results suggest that circulating IL-1β is a surrogate for protein locally produced in the hind limb.

elevation compared to baseline significant p<0.05.

FIG. 10. Determination of IL-1α mRNA expression in 30, 40, or 60 Gy irradiated limbs from 3 mice strains by RNase protection assay. Each value was normalized to its internal control GAPDH and represents the pooled expression from seven mice per measurement. Radiation elevated IL-1α mRNA in skin (a and b) but not in muscle tissue (c). The effect was greater with increased radiation dose. C3H/HeN mice express no detectable IL-1α mRNA in their skin at any time after irradiation.

Elevation of IL-1α during the first day after radiation was most pronounced in the fibrosis sensitive strain.

FIG. 11. Determination of IL-1Ra mRNA expression in 30, 40, or 60 Gy irradiated limbs from 3 mice strains by RNase protection assay. Each value was normalized to its internal control L32 and represents the pooled expression from seven mice per measurement. Radiation-dose and time dependant induction of IL-1Ra mRNA mainly occurred in skin, with no detectable induction in muscle tissue. The fibrosis sensitive strain had the greatest induction of IL-1Ra.

### REFERENCES

1. H. D. Suit, Radiation biology: the conceptual and practical impact on radiation therapy. Radiat Res 94, 10-40 (1983).

2. H. D. Suit and R. Miralbell, Potential impact of improvements in radiation therapy on quality of life and survival. Int J Radiat Oncol Biol Phys 16, 891-5 (1989).

3. H. Suit, S. Skates, A. Taghian, P. Okunieff and J. T. Efird, Clinical implications of heterogeneity of tumor response to radiation therapy. Radiother Oncol 25, 251-60 (1992).

4. H. D. Suit and A. M. Walker, Assessment of the response of tumours to radiation: clinical and experimental studies. Br J Cancer Suppl 41, 1-10 (1980).

5. J. L. Lefaix, S. Delanian, B. Dubray, P. Giraud, E. Berland, S. Sahraoui and J. Mignot, [Changes of the cutaneous micro-relief in superficial radiation-induced fibrosis: a qualitative study]. Bull Cancer 83, 915-22 (1996).

6. J. L. Lefaix, O. Verola, C. Brocheriou and F. Daburon, Early changes in irradiated skeletal muscle: a histo-enzymological and immunocytochemical study. Br J Radiol Suppl 19, 109-13 (1986).

7. H. D. Suit, G. Silver, R. S. Sedlacek and A. Walker, Experimental condition and the acute reaction of mouse skin to ionizing radiation. Radial Res 95, 427-33 (1983).

8. H. P. Rodemann, H. P. Peterson, K. Schwenke and K. H. von Wangenheim, Terminal differentiation of human fibroblasts is induced by radiation. Scanning Microsc 5, 1135-42; discussion 1142-3 (1991).

9. M. Martin, J. Lefaix and S. Delanian, TGF-betal and radiation fibrosis: a master switch and a specific therapeutic target? Int J Radiat Oncol Biol Phys 47, 277-90 (2000).

10. M. Martin, J. Remy and F. Daburon, Abnormal proliferation and aging of cultured fibroblasts from pigs with subcutaneous fibrosis induced by gamma irradiation. J Invest Dermatol 93, 497-500 (1989).

11. K. E. McGrath, A. D. Koniski, K. M. Maltby, J. K. McGann and J. Palis, Embryonic expression and function of the chemokine SDF-1 and its receptor, CXCR4. Dev Biol 213, 442-56 (1999).

12. C. L. Dileto and E. L. Travis, Fibroblast radiosensitivity in vitro and lung fibrosis in vivo: comparison between a fibrosis-prone and fibrosis-resistant mouse strain. Radiat Res 146, 61-7 (1996).

13. M. W. Skwarchuk and E. L. Travis, Changes in histology and fibrogenic cytokines in irradiated colorectum of two murine strains. Int J Radiat Oncol Biol Phys 42, 169-78 (1998).

14. P. Okunieff P. Rubin, J. Williams, J. Finkelstein and l. Ding, TGF-β1 is an important contributing factor in the development of radiation induced fibrosis. Int. J. Radiat. Oicol. Biol. Phys. Suppl 48, 246 (2000).

15. M. S. Anscher, I. R. Crocker and R. L. Jirtle, Transforming growth factor-beta 1 expression in irradiated liver. Radiat Res 122, 77-85 (1990).

16. M. S. Anscher, W. P. Peters, H. Reisenbichler, W. P. Petros and R. L. Jirtle, Transforming growth factor beta as a predictor of liver and lung fibrosis after autologous bone marrow transplantation for advanced breast cancer. N Engl J Med 328, 1592-8 (1993).

17. M. S. Anscher, T. Murase, D. M. Prescott, L. B. Marks, H. Reisenbichler, G. C. Bentel, D. Spencer, G. Sherouse and R. L. Jirtle, Changes in plasma TGF beta levels during pulmonary radiotherapy as a predictor of the risk of developing radiation pneumonitis. Int J Radiat Oncol Biol Phys 30, 671-6(1994).

18. F. M. Kong, M. S. Anscher, T. Murase, B. D. Abbott, J. D. Iglehart and R. L. Jirtle, Elevated plasma transforming growth factor-beta 1 levels in breast cancer patients decrease after surgical removal of the tumor. Ann Surg 222, 155-62 (1995).

19. M. S. Anscher, F. M. Kong and R. L. Jirtle, The relevance of transforming growth factor beta 1 in pulmonary injury after radiation therapy. Lung Cancer 19, 109-20 (1998).

20. L. B. Marks, M. Fan, R. Clough, M. Munley, G. Bentel, R. E. Coleman, R. Jaszczak, D. Hollis and M. Anscher, Radiation-induced pulmonary injury: symptomatic versus subclinical endpoints. Int J Radiat Biol 76, 469-75 (2000).

21. C. J. Johnston, T. W. Wright, P. Rubin and J. N. Finkelstein, Alterations in the expression of chemokine mRNA levels in fibrosis- resistant and -sensitive mice after thoracic irradiation. Exp Lung Res 24, 321-37 (1998).

22. I. Ding, X. Q. Gong, M. Li, Y. Tagaya, N. Azimi and P. Okunieff, Radiation mediated interleukine-15 production in normal and malignant cell lines. Radiation Res 46th Annual Proceedings. pp 147 (1998).

23. I. Ding, X. Q. Gong, M. Li, W. H. Xu, D. Tang, R. Greig and P. Okunieff, TGFβ and FGF2 mRNA and protein expression in normal and malignant cells after ionizing radiation. Radiation Res 46th Annual Procedings. pp 147 (1998).

24. C. K. Haston and E. L. Travis, Murine susceptibility to radiation-induced pulmonary fibrosis is influenced by a genetic factor implicated in susceptibility to bleomycin-induced pulmonary fibrosis. Cancer Res 57, 5286-91 (1997).

25. R. Neta, S. Douches and J. J. Oppenheim, Interleukin 1 is a radioprotector. J Immunol 136, 2483-5 (1986).

26. R. Neta, J. J. Oppenheim, J. M. Wang, C. M. Snapper, M. A. Moorman and C. M. Dubois, Synergy of IL-1 and stem cell factor in radioprotection of mice is associated with IL-1 up-regulation of mRNA and protein expression for c-kit on bone marrow cells. J Immunol 153, 1536-43 (1994).

27. Engelstad, R B: Pulmonary lesions after roentgen and radium irradiation. Am J Roentgen 43:676-681, 1940.

28. Roberts C M, Foulcher E, Zaunders J J, et al: Radiation pneumonitis: A possible lymphocyte-mediated hypersensitivity reaction. Ann Intern Med 118:696-700, 1993.

29. Gross, N J: Pulmonary effects of radiation therapy. Ann Intern Med 86:81-92, 1977.

30. Fryer C J H, Fitzpatrick P J, Rider W D et al: Radiation pneumonitis: experience following a large single dose of radiation. Int J Radiat Oncol Biol Phys 4:931-936, 1978.

31. Chen Y, Rubin P, Williams J, et a: Circulating IL-6 as a predictor of radiation pneumonitis. Int J Radiat Oncol Biol Phys 49:641-648, 2001.

32. Anscher M S, Kong F-M, Andrews K, et al: Plasma transforming growth factor β1 as a predictor of radiation pneumonitis. Int J Radiat Oncol Biol Phys 41:1029-1035, 1998.

33. Rubin P, Johnston C J, Williams J P, et al: A perpetual cascade of cytokines post-irradiation leads to pulmonary fibrosis. Int J Radiat Oncol Biol Phys 33:99-109, 1995.

34. Johnston C J, Wright T W, Rubin P, et al. Alterations in the expression of chemokine mRNA levels in fibrosis-resistant and -sensitive mice after thoracic irradiation. Exp Lung Res 24:321-337, 1998.

35. Kaseda M, Kadota J, Mukae H, et al: Possible role of L-selectin in T lymphocyte alveolitis in patients with active pulmonary sarcoidosis. Clin Exp Immunol 121:146-150, 2000.

36. Butcher E C, Picker L J I: Lymphocyte homing and homeostasis. Science 272:60-66, 1996.

37. McDonald S, Rubin P, Phillips T L, et al: Injury to the lung from cancer therapy: clinical syndromes, measurable endpoints, and potential scoring systems. Int J Radiat Oncol Biol Phys 31:1187-1203, 1995.

38. Antonia S J, Wagner H, Williams C, et al: Concurrent paclitaxel/cisplatin with thoracic radiation in patients with stage III A/B non-small cell carcinoma of the lung. Semin Oncol 22:34-37, 1995.

39. Reckzeh B, Merte H, Pflluger K-H, et al: Severe lymphocytopenia and interstitial pneumonia in patients treated with paclitaxel and simultaneous radiotherapy for non-small cell lung cancer J Clin Oncol 14:1071-0176, 1996.

40. Aitonadou D, Coliarakis N, Synodinou M, et al: Randomized phase III trial of radiation±amifostine in patients with advanced stage lung cancer. Int J Radiat Oncol Biol Phys 45(S): 154, 1999.

41. Schuyler M, Gott K, Cherne A: Mediators of hypersensitivity pneumonitis. J Lab Clin Med 136:29-38, 2000.

42. Ding z, Xiong K, Issekutz T B: Regulation of chemokine-induced transendothelial migration of T lymphocytes by endothelial activation: differential effects on naive and memory T cells. J Leukoc Biol 67:825-833, 2000.

43. Maasilta P, Hallman M, Taskinen E. et al: Bronchoalveolar lavage fluid findings following radiotherapy for non-small cell lung cancer. Int J Radiat Oncol Biol Phys 26:117-123; 1993.

44. De Boer W I, Sont J K, van Schadewijk A, et al: Monocyte chemoattractant protein I, interleukin 8, and chronic airways inflammation in COPD. J Pathol 190:619-626, 2000.

45. Kovacs E J: Fibrogenic cytokines: the role of immune mediators in the development of scar tissue. Immunol Today 12:17-23, 1991.

46. Elias J A, Schreiber A D, Gustilo K, et al: Differential interleukin-1 elaboration by unfractionated and density fractionated human alveolar macrophages and blood monocytes: relationship to cell maturity. J Immunol 135:3198-3204, 1985.

47. Kotloff R M, Little J, Elias J A: Human alveolar macrophage and blood monocyte interleukin-6 production. Am J Respir Cell Mol Biol 3:497-505, 1990.

48. Simeonova P P, Toriumi W, Komnmineni C, et al: Molecular regulation of IL-6 activation by asbestos in lung epithelial cells: role of reactive oxygen species. J Immunol. 159:3921-3928, 1997.

49. O'Brien-Lander A, Nelson M E, Kimler B F, et al: Release of Interleukin-1 by human alveolar macrophages after in vitro irradiation. Radiat Res 136:37-41, 1993.

50. Elias J A, Lentz V: IL-1 and tumor necrosis factor synergistically stimulate fibroblasts IL-6 production and stabilize IL-6 messenger RNA. J Immunol 145:161-166, 1990.

51. Helle M, Brakenhoff J P J, De Groot E R et a:. Interleukin 6 is involved in interleukin 1-induced activities. Eur J Immunol 18:957-959, 1988.

52. Roach M III, Gandara D R, Yuo H S, et al: Radiation pneumonitis following combined modality therapy for lung cancer: Analysis of prognostic factors. J Clin Oncol. 13:2606-2612, 1995.

53. Kimsey F C, Mendenliall N P, Ewald L M, et al: Is radiation treatment volume a predictor for acute or late effect on pulmonary function? Cancer 73:2549-2555, 1994.

54. Byhardt R W, Martin L, Pajak T F et al: The influence of field size and other treatment factors on pulmonary toxicity following hyperfractionated irradiation for inoperable non-small cell lung cancer (NSCLC)-Analysis of a Radiation Therapy Oncology Group (RTOG) Protocol. Int J Radiat Oncol Biol Phys 27:537-544, 1993.

55. Kwa S L S, Lebesque J V, Theuws J C M et al:, Radiation pneumonitis as a function of mean lung dose: an analysis of pooled data of 540 patients. Int J Radiat Oncol Biol Phys 42:1-9, 1998.

### EXAMPLE 3

Material and Methods

Tumor Models and Radiation Treatment

Isotransplantable murine MCa-35 mammary tumor cells was inoculated i.m. into right hind thighs of 6-7 week-old female C3H/HeN mice (NCI, Fredrick, Md.). Right hind thigh tumors were given 60 Gy (single dose using a Cs irradiator) when tumors reached 8-9 mm in diameter. Mice were sacrificed 20 days after radiation.

Tumors and the overlaying skin tissues were removed for histology and RNA preparation. Irradiated tissues (tumor and skin) were also collected for making paraffin blocks for immunohistochemical staining. Guidelines for the humane treatment of animals were followed as approved by the University of Rochester Committee on Animal Resources.

Celebrex Treatment

Celebrex (Pfizer Inc.) powder was dissolved in PBS. Due to partial dissolution, the agent was mixed very well every time before gavaging. 50 mg/kg (0.2 ml) Celebrex was given daily, and five days per week for constitutive three weeks. Four experiment groups were used. All mice were treated with single 60 Gy radiation in tumor-bearing leg. Group 1 was radiation alone; mice in group 2 were given 50 mg/kg Celebrex 2 hours before radiation (2 hr pre-radiation); mice in group 3 and 4 were received the same amount of Celebrex at day 2 or day 7 post-radiation. Mice in the group 4 were received total 10 doses, and rest treated mice were given total 15 doses. All mice were sacrificed 20 day after radiation.

Determination of Radiation Induced Skin Damage by 5-Scales Scoring System

Radiation induce skin damage was assessed using 5-scales Skin Scoring System. 20 days after single 60 Gy radiation, mice from each treatment group were determined blindly for the degree of skin damage by three investigators. Grade 1: normal skin; grade 2: slight hair loss in irradiated area; grade 3: radish and swollen tissue; grad 4: small area erosion; grade 5: small ulceration. Grades 2-3 is referred as mild, and grades 4-5 is considered as severe skin damage.

Tumor Tissue RNA Isolation and RNase Protection Assays

Total RNA was isolated from tumors and skin overlying tumors, respectively, with 9-10 mice in each treatment group by pulverizing the frozen tissue, and dissolved in TRI Reagent (Molecular Research Center, Ohio) according to the manufacturer's specifications. To determine the integrity of isolated RNA, 2 μg of RNA from each sample was fractionated on a formaldehyde gel and visualized by staining in ethidium bromide. RNase protection was performed using established multi-probe template sets (PharMingen, San Diego, Calif.) as described previously [Okunieff, 1998 #4388]. The chemokine multiple templet includes: MCP-1, MIP-1α, MIP-1β, MIP-2, Rantes, Eotaxin and IP-10. The C-C chemokine receptor multiple templete includes: CCR1, CCR2, CCR3, CCR4 and CCR5. The C-X-C chemokine receptor multiple templets includes: CXCR2 and CXCR4. Two internal standards, L32 and GAPDH, were used as loading controls. The cocktail constructs were used to prepare ³²P-UTP labeled antisense cDNA probes using PharMingen in vitro transcription kits (PharMingen, San Diego, Calif.). Probes were hybridized with 30 μg of total RNA at 50° C. for 16 hrs RNase A (1 mg/ml), and RNase T1 (2000 U/ml) was then added to digest single-stranded RNA. After digestion, the RNA was precipitated and resuspended in gel loading buffer, heated at 95° C. for 5 min, and run on a 6M urea, 7% denaturing polyacrylamide gel (National Diagnostics, Ga.). The gel was dried on filter paper and placed on a phosphorimager screen for quantitative analysis of mRNA expression levels for each cytokine/chemokine. Area integration of each mRNA-protected fragment probe was normalized against the protected band for GAPDH or L32 mRNA in each corresponding lane to calculate the ratio of targeted mRNA/GAPDH mRNA expression. In order to compare the basal levels of each gene tested, relative levels (ratios) were plotted.

Quantitative Measurement of Total Structural and Perfused Vessels

Immunohistochemistry methods have previously been described in detail. Immediately following cryostat sectioning, tissue slices (normal muscle and tumor) were stained with CD31 antibody (PharMingen Calif.) for determination of total vasculature. The stained sections were imaged using an epi-fluorescence equipped microscope, digitized (3-CCD camera), background-corrected, and image-analyzed using Image Pro software (Media Cybernetics, Mass.) and a 450 MHz Pentium computer. Color images from individual microscope fields were automatically acquired and digitally combined to form four montages of the tumor cross-section (total area=15.5 mm2) using a motorized stage and controller. The image montages were processed to enhance the contrast between background and CD31 staining. From the enhanced images, locations of CD31-stained vessels were recorded. The quantitative vascular information was analyzed using custom Fortran programs to perform a “closest individual” analysis as previously described. Briefly, the distances from computer-superimposed sampling points to the nearest blood vessel were determined. The cumulative frequency distribution of these distances provided the probability of encountering vessels within any specified distance from the tumor cells. Median distances (μm) to the nearest vessel were used for statistical comparisons.

Statistical Analysis

mRNA levels (ratios) of tumors and skin from irradiated or non-irradiated mice were evaluated using the unpaired Students t-test or Mann-Whitney Rank Sum test as appropriate. Differences were considered significant for p<0.05.

Results

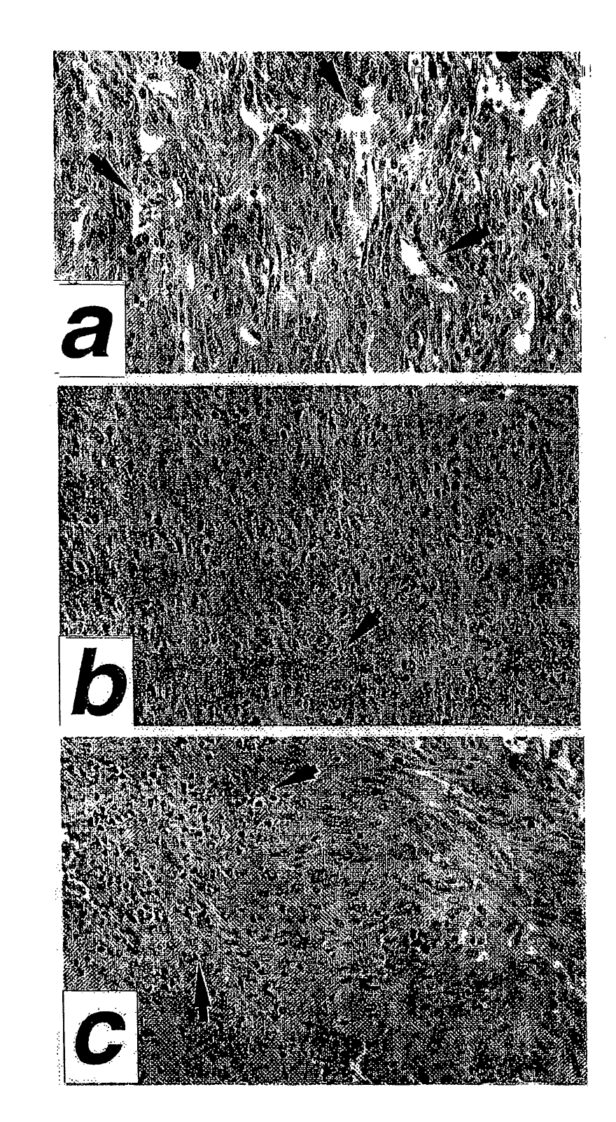

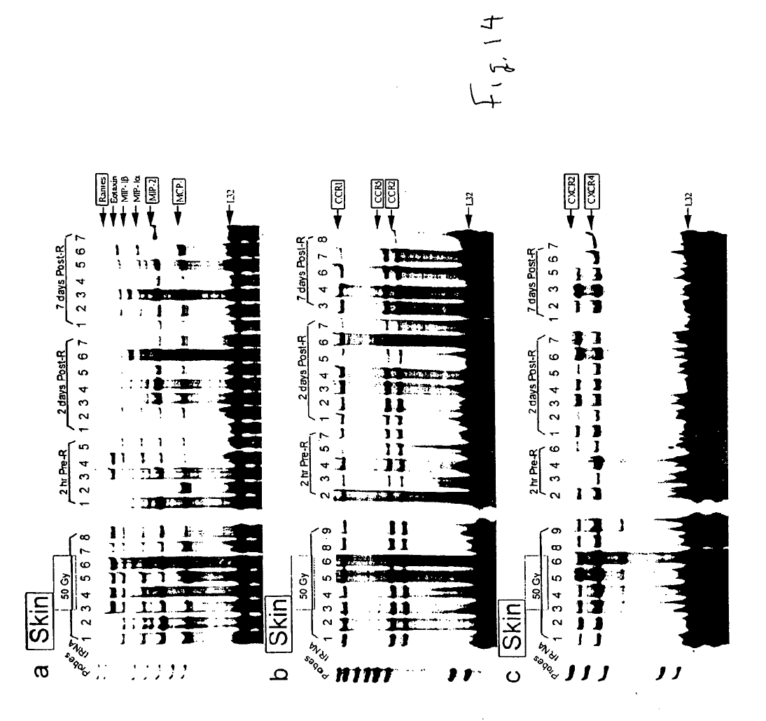

20 days after single 60 Gy irradiated MCa-35 tumor skin had varied lesions including edema, erosion and superficial necrosis in most of saline-treated control mice 20 days after radiation (FIG. 12a). However, Celebrex-treated tumors had less radiation-induced skin damage compared with saline-treated controls (FIG. 12b-d). The most of Celebrex treated mice, regardless pre- (2 hr before radiation) or post-radiation (day 2 or day 7 after radiation) had less inflammation and cellular component infiltration in the dermis (FIG. 13b, c and d) compared with saline-treated controls (FIG. 13a). 23.8% (5/21) of mice in 60 Gy alone treated group developed severe skin damage, but only 17.6% of mice in the pre-2hr Celebrex treated group, 5.3% of mice in the post-day 2 Celebrex treated group, and 11.1% of mice in post-day 7 Celebrex-treated group, appeared as the severe skin damage 20 days after radiation. Oral administration of Celebrex also caused the reduction of blood vessels in MCa-35 tumor (FIG. 12f), focal necrosis (FIG. 12g) and even massive tumor necrosis (FIG. 12h) in some areas of tumors, compared with saline-treated controls (FIG. 12e).

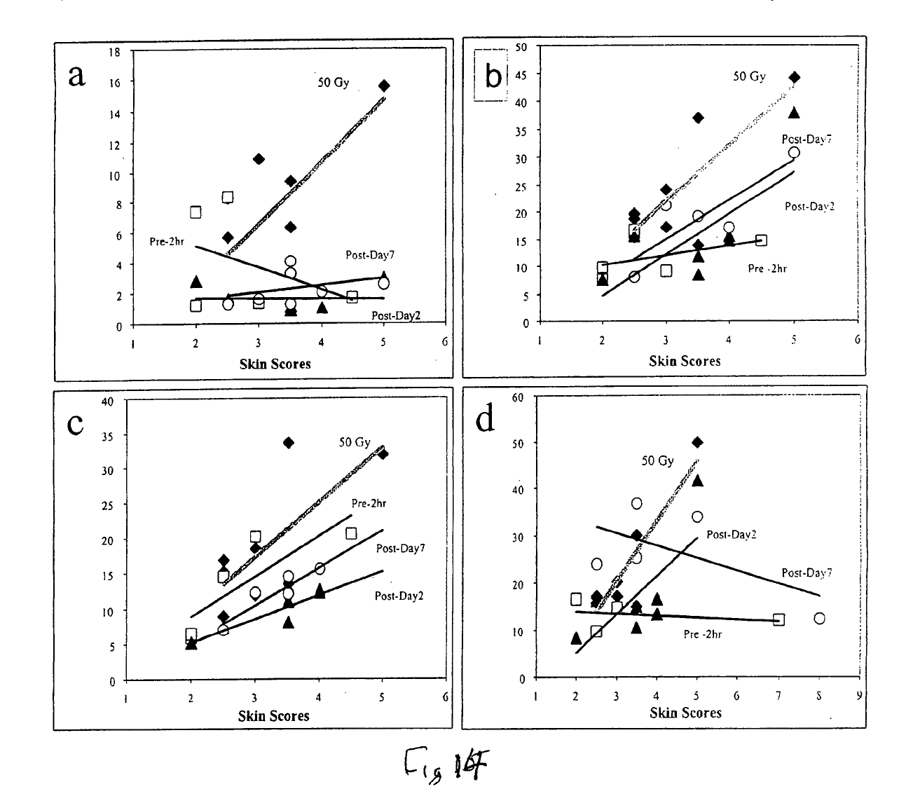

Because radiation inducing soft tissue damages has been reported to associate with the persistent overproduction of cytokine or chemokine in irradiated normal or tumor cells, we next examined the effects of Celebrex on the radiation-induced mRNA expression of chemokines including five C-C family members (Rantes, eotactin, MIP-1α, MIP-β and MCP-1), one C-X-C family (MIP-2) and one C family member (lymphotactin), as well as C-C receptors (CCR1, CCR2 and CCR5) and C-X-C receptors (CXCR2 and CXCR4) in tumor skin and tumor tissues by RNase protection assay. As shown in FIG. 14 and summarized in Table 3 and 4, Celebrex treatment caused the significant reduction of Rantes (2.3±1.1 vs 7.4±1.6, P<0.05) and MCP-1 (10.2±1.1 vs 18.8±3.2, p<0.05) mRNA expression in irradiated skin tissues, but not in tumor tissues (Table 3), Although radiation induced higher levels of skin MIP-2 mRNA expression in 37.5% (⅜) of mice, only 14.3%-28.6% of tumor skin had high MIP-2 mRNA expression after Celebrex treatment. Similarly, Celebrex-treatment did not significantly alter the tumor MIP-2 mRNA (Table 4). Celebrex not only reduced C-C chemokines, it also caused the decrease mRNA expression of both C-C and C-X-C chemokine receptors in tumor skin (FIG. 14B and D), not in tumor tissues (FIG. 14C). All quantitative measurement are shown in Tables 3 and 4.

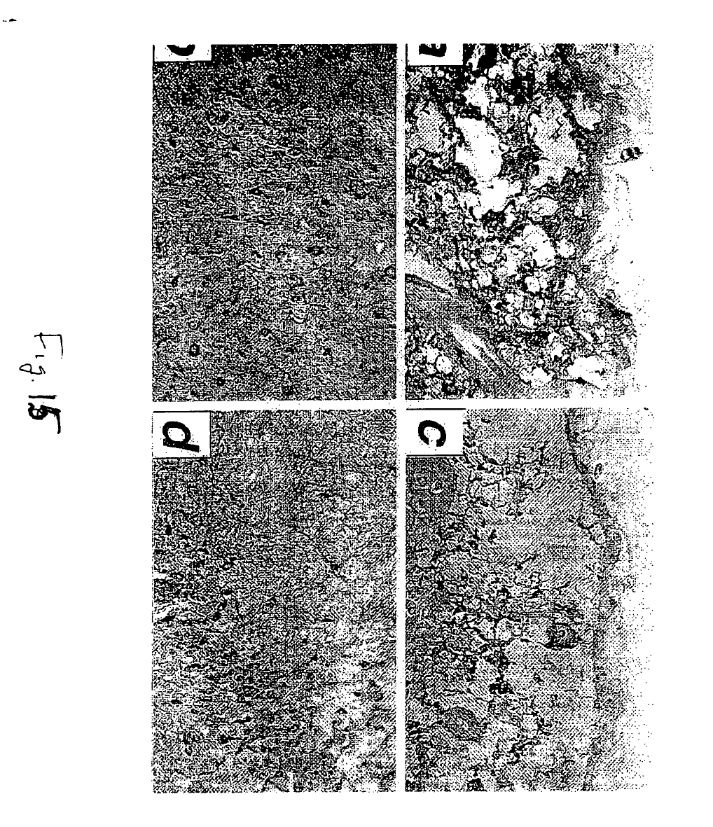

Due to each individual mouse variation, there was 15-30% of Celebrex-treated mice still developed the moderate or severe skin damage after radiation. Radiation-induced skin damage was quantitatively determined by the skin scores from each individual mouse. In order to find out the relationship between overexpression of chemokines or their receptors mRNA and radiation-induced skin damages, the correlation of skin scores and skin tissue chemokine and chemokine receptor mRNA expression levels from each individual mouse were plotted and shown in FIG. 15. Significant positive correlation between skin damages (skin scores) and overexpression of chemokine and its receptor mRNA expression were observed in 60 Gy radiation-treated mice. However, the correlation of Celebrex-mediated the reduction of chemokine and chemokine receptor mRNA expression with skin damages only occurred in Rantes (FIG. 15a) and it receptor CCR5 (FIG. 15d), MCP-1 (FIG. 15b) and its receptor CCR2 (FIG. 15d). Although Celebrex-mediated reduction of MIP-2 mRNA expression did not correlate with less skin damage, related CXCR4 mRNA expression was significantly reduced in Celebrex-treated mice, which had less radiation-induced skin damage.

As shown in FIG. 16, Celebrex-treated mice had less infiltration of inflammatory cells in the dermas (FIG. 16c) compared with saline controls (FIG. 16a). However, the infiltration of inflammatory cells in tumor tissue was not obviously altered by Celebrex treatment (FIG. 16b and d).

Discussion

Thus we have discussed that: 1) Radiation induced Rantes/CCR5 and MCP-1/CCR2 mRNA expression was decreased by Celebrex; and 2) Celebrex-mediated reduction of chemokine and their receptor mRNA expression was correlated with ameliorated skin damage. 2

We claim:

1. A method for prophylactically treating radiation toxicity in normal tissue of a subject comprising administering an anti-radiation toxicity effective amount of a cytokine blocking agent to said subject.

2. The method of claim 1 wherein said cytokine blocking agent is administered to said subject prior to said subject receiving radiation therapy.

3. The method of claim 1 wherein said cytokine blocking agent is administered to said subject simultaneously with radiation therapy.

4. The method of claim 1 wherein said cytokine blocking agent comprises anakinra or a MCP-1 blocker or a TGF-beta blocker.

5. The method of claim 1 wherein said cytokine blocking agent is administered orally or parentally.

6. The method of claim 6 wherein said parental administration is by intravenous infusion.

7. The method of claim 1 wherein said cytokine blocking agent blocks activity of cytokine IL-1.

8. The method of claim 1 wherein said cytokine blocking agent blocks activity of IL-1α or IL-1β.

9. A method for prophylactically treating radiation toxicity in normal tissue of a subject comprising regulating cytokine IL-1 activity in said subject to decrease the radiation toxicity.

10. A method for prophylactically treating radiation pneumonitis, dermatitis, soft tissue fibrosis or central nervous system toxicity in a subject comprising administering an anti-radiation pneumonitis effective amount, an anti-radiation dermatitis effective amount, an anti-soft tissue fibrosis effective amount, or an anti-central nervous system toxicity effective amount of a cytokine blocking agent to said subject.

11. A method for prophylactically treating radiation pneumonitis, dermatitis, soft tissue fibrosis or central nervous system toxicity in a subject comprising administering an anti-radiation pneumonitis effective amount, an anti-radiation dermatitis effective amount, an anti-soft tissue fibrosis effective amount, or an anti-central nervous system toxicity effective amount of anakinra, a MCP-1 blocker, or a TGF-beta blocker.

12. A method for treating radiation toxicity in normal tissue of a subject comprising administering an anti-radiation toxicity effective amount of a cytokine blocking agent to said subject subsequent to subjecting said subject to radiation therapy.

13. A method for diagnosing the likelihood for the occurrence of radiation toxicity in the normal tissue of a subject comprising measuring the amount of cytokine IL-1, IL-160 , IL-1β or IL-6 cytokines in the subject, the relative amount of such activity being indicative of the likelihood of the subject experiencing an adverse degree of radiation toxicity when subjected to therapeutic radiation.

14. A method for prophylactically treating radiation toxicity in the normal tissue of a subject comprising first diagnosing the likelihood for the occurrence of radiation toxicity in the normal tissue of a subject comprising measuring the amount of cytokine IL-1, IL-1α, IL-1β or IL-6 cytokines in the subject, the relative amount of such activity being indicative of the likelihood of the subject experiencing an adverse degree of radiation toxicity when subjected to therapeutic radiation and then administering an anti-radiation toxicity effective amount of a cytokine blocking agent to said subject diagnosed as being likely to experience an adverse degree of radiation toxicity when subjected to therapeutic radiation.
